(19) United States
(12) Patent Application Publication
(10) Pub. No.: US 2024/0361306 A1
(43) Pub. Date: Oct. 31, 2024

(54) COMPOSITIONS FOR IMMUNOLABELING

(71) Applicant: Cellstate Biosciences Inc., Oro Valley, AZ (US)

(72) Inventors: Arron Sullivan, Tucson, AZ (US); Andrew P. Capaldi, Tucson, AZ (US); Roderick Capaldi, Eugene, OR (US); Nikita Fernandes, Oro Valley, AZ (US)

(21) Appl. No.: 18/648,147

(22) Filed: Apr. 26, 2024

Related U.S. Application Data
(60) Provisional application No. 63/498,472, filed on Apr. 26, 2023.

(51) Int. Cl. G01N 33/542; C07K 16/00

(52) U.S. Cl. CPC G01N 33/542; C07K 16/00; C07K 2317/569; C07K 2317/622; C07K 2319/60

(57) ABSTRACT

Isolated antibody compositions for immunolabeling are described herein. These compositions overcome many of the limitations currently involved with immunolabeling such as low fluorescence and high noise signal.

### CROSS-REFERENCE TO RELATED APPLICATIONS

This application is a non-provisional and claims benefit of U.S. Provisional Application No. 63/498,472 filed Apr. 26, 2023, the specification of which is incorporated herein in their entirety by reference.

### FIELD OF THE INVENTION

The present invention features isolated antibody compositions and synthetic binder compositions for immunolabeling.

### BACKGROUND OF THE INVENTION

Immunolabeling is a method for detecting and localizing an antigen to a site within a cell, organ, or tissue. Immunolabeling procedures were established during the 1940s with the development of antibodies (immunoglobulins, Ig) for specific antigens (e.g., protein) and are widely used in neuroscience and cell biology research. They have enabled important discoveries of molecular and cellular processes in both normal and disease states to be made.

Both direct and indirect labeling of antibodies can be used for immunolabeling. With direct labeling, only a primary antibody (e.g., an antibody specific to an antigen) bound to the label is used. In contrast, a secondary antibody bound to the label, which is specific for the primary antibody, is used with indirect labeling. However, despite being established for over 50 years, both direct and indirect labeling still have limitations. For example, direct immunolabeling relies on the number of fluorophores attached to a single antibody and, consequently, can limit detection to high-abundance targets. Additionally, indirect immunolabeling relies on using secondary antibodies (usually polyclonal) that have off-target noise and require multiple primary and secondary host species for multiplexing.

The present invention overcomes these limitations and provides antibody compositions with brighter fluorescence.

### BRIEF SUMMARY OF THE INVENTION

It is an objective of the present invention to provide compositions that allow for singleplex and/or multiplex immunolabeling, as specified in the independent claims. Embodiments of the invention are given in the dependent claims. Embodiments of the present invention can be freely combined with each other if they are not mutually exclusive.

The present invention features synthetic binder compositions. In some embodiments, the synthetic binder composition may include a primary target binder configured to directly bind to a target (e.g., a protein) and a reporter (or drug molecule) conjugate to at least one conjugation-targeted amino acid residue (e.g., a cysteine residue, a lysine residue, or a combination thereof) in the primary target binder. In other embodiments, the synthetic binder composition may include a primary target binder configured to directly bind to a target (e.g., a protein) and a reporter (or drug molecule) conjugated to at least one post-translational modification (e.g., a glycosylation moiety). In further embodiments, the synthetic binder composition may include a primary target binder configured to directly bind to a target (e.g., a protein) and one or a combination of: (i) a reporter conjugated to at least one conjugation-targeted amino acid residue in the primary target binder; (ii) a drug conjugated to at least one conjugation-targeted amino acid residue in the primary target binder; (iii) a reporter conjugated to at least one post-translational modification on the primary target binder; or (iv) a drug conjugated to at least one post-translational modification on the primary target binder.

In some embodiments, the conjugation-targeted amino acid residues throughout the primary binder are spaced such that when the reporter (or drug molecule) is conjugated to the conjugation-targeted amino acid residue, one reporter (or drug molecule) does not interfere with the conjugation of another reporter (or drug molecule). In some embodiments, the reporter (or drug molecule) is conjugated to an amino acid residue that is naturally occurring within the primary target binder sequence. In other embodiments, the reporter (or drug molecule) is conjugated to an amino acid that has been modified (e.g., substituted or added) within the primary target binder sequence. For example, the primary target binder described herein may be engineered for optimized conjugation of the reporter (or drug molecule). In some embodiments, the primary target binder includes an antibody or a fragment thereof. The antibody fragment may include a fragment antigen-binding region (Fab region), F (ab′)2, Fab′, Fv (Fragment Variable), single-domain antibody (sdAb) fragment, a single-chain variable fragment (scFv), or the like. In other embodiments, the primary target binder includes a synthetic binder or an engineered binder. The synthetic binder may include an aptamer, affibody, Designed Ankyrin Repeat Protein (DARPins), molecularly imprinted polymer, or the like. In some embodiments, the reporter is a fluorescence reporter (e.g., fluorescence dye), fluorochrome, or an enzyme reporter. Non-limiting examples of enzyme reporters may include a chromogen such as 3,3′-diaminobenzidine tetrahydrochloride (DAB), horseradish peroxidase (HRP), or alkaline phosphatase (AP). In other embodiments, the reporter is an oligonucleotide or a metal isotope.

Alternatively, the present invention may feature a synthetic binder composition comprising a) a primary target binder configured to directly bind a target molecule, b) a primary fusion component attached (e.g., at either a C or N terminal) to the primary target binder, and one or a combination of: (i) a reporter conjugated to at least one conjugation-targeted amino acid residue in either one or both of the primary target binder or the primary fusion component; (ii) a drug conjugated to at least one conjugation-targeted amino acid residue in either one or both of the primary target binder or the primary fusion component; (iii) a reporter conjugated to at least one post-translational modification (e.g., a glycosylation moiety) on one or both of the primary target binder or the primary fusion component; or (iv) a drug conjugated to at least one post-translational modification (e.g., a glycosylation moiety) on one or both of the primary target binder or the primary fusion component. In some embodiments, the primary fusion component is recombinantly linked to the primary target binder. In other embodiments, the primary fusion component binds directly to the primary target binder. The primary fusion composition may either be (i) a binding component that allows binding of a secondary binder to the primary target binder (e.g., an epitope or a pseudo-epitope) or (ii) a secondary binder. In certain embodiments featuring the primary fusion component as a secondary binder, the secondary binder may either be recombinantly linked to the primary target binder or directly bind to it. In further embodiments, the primary target binder and/or primary fusion component may further comprise a glycosylation that a reporter is conjugated to.

In some embodiments, the present invention features an isolated antibody composition comprising a constant region (Fc) and a variable region (Fab). The constant region may include one or more amino acid modifications (e.g., substitutions, deletions, additions), wherein at least one of the one or more amino acid modifications (e.g., substitutions) includes an amino acid modified (e.g., substituted) to a lysine or cysteine residue. In some embodiments, the variable region includes one or more amino acid modifications (e.g., substitutions), wherein at least one of the one or more modifications (e.g., substitutions) includes an amino acid modified (e.g., substituted) to a lysine or cysteine residue. In some embodiments, a conjugation occurs at at least one of lysine or cysteine residues. In some embodiments, naturally occurring and substituted lysine or cysteine residues throughout the constant region are spaced such that when there is a conjugation to a lysine or cysteine residue it has limited interference with conjugations on another lysine or cysteine residue.

In some embodiments, the present invention features an isolated antibody composition comprising a constant region (Fc) and a variable region (Fab). The constant region may include one or more amino acid modifications (e.g., substitutions), wherein at least one of the one or more amino acid modifications (e.g., substitutions) includes an amino acid modified (e.g., substituted) to a lysine or cysteine residue. In some embodiments, a reporter is conjugated to at least one of the lysine or cysteine residues. In other embodiments, a drug molecule is conjugated to at least one of the lysine or cysteine residues. Naturally occurring and substituted lysine or cysteine residues throughout the constant region may be spaced such that when the reporter (or drug molecule) is conjugated to a lysine or cysteine residue, one reporter (or drug molecule) does not interfere with the conjugation of another reporter (or drug molecule).

In some embodiments, the present invention features an isolated antibody composition comprising at least one variable region (Fab) comprising a light chain and a portion of a heavy chain (e.g., a heavy chain or a portion thereof), and a protein recombinantly linked to the C-terminal of the heavy chain or the light chain. The protein may be natural, synthetic, or a combination thereof and include one or more amino acid modifications (e.g., substitutions), wherein at least one of the one or more amino acid modifications (e.g., substitutions) includes an amino acid modified (e.g., substituted) to a lysine or cysteine residue. In some embodiments, a reporter is conjugated to at least one of the lysine or cysteine residues. In other embodiments, a drug molecule is conjugated to at least one of the lysine or cysteine residues. Naturally occurring and substituted lysine or cysteine residues throughout the constant region may be spaced such that when the reporter (or drug molecule) is conjugated to a lysine or cysteine residue, one reporter (or drug molecule) does not interfere with the conjugation of another reporter (or drug molecule).

In other embodiments, the present invention features an isolated antibody composition comprising at least one variable region (Fab) comprising a light chain and a portion of a heavy chain (e.g., a full-length heavy chain or a portion thereof), and a protein recombinantly linked to the portion (e.g., a C-terminal or N-terminal) of the heavy chain. In some embodiments, a reporter is conjugated to at least one of the lysine or cysteine residues. In other embodiments, a drug molecule is conjugated to at least one of the lysine or cysteine residues. The lysine or cysteine residues throughout the protein are space such that when a reporter (or drug molecule) is conjugated to a lysine or cysteine residue, one reporter (or drug molecule) does not interfere with the conjugation of another reporter (or drug molecule).

In some embodiments, the present invention features an isolated antibody composition comprising an antibody (AB) fragment and a protein is recombinantly linked to the AB fragment. In some embodiments, a reporter is conjugated to at least one of the lysine or cysteine residues. In other embodiments, a drug molecule is conjugated to at least one of the lysine or cysteine residues. The lysine or cysteine residues throughout the protein are space such that when a reporter (or drug molecule) is conjugated to a lysine or cysteine residue, one reporter (or drug molecule) does not interfere with the conjugation of another reporter (or drug molecule).

In other embodiments, the present invention features an isolated binder composition comprising a synthetic binder and a protein is recombinantly linked to the synthetic binder. In some embodiments, a reporter is conjugated to at least one of the lysine or cysteine residues. In other embodiments, a drug molecule is conjugated to at least one of the lysine or cysteine residues. The lysine or cysteine residues throughout the protein are space such that when a reporter (or drug molecule) is conjugated to a lysine or cysteine residue, one reporter (or drug molecule) does not interfere with the conjugation of another reporter (or drug molecule). In other embodiments, a reporter or drug molecule is conjugated to a glycosylation or other post-translational modification.

In alternative embodiments, the antibody compositions or recombinantly added proteins described herein do not comprise sequence tailoring with one or more amino acid modifications (e.g., substitutions, additions, or deletions).

One of the unique and inventive technical features of the present invention is the use of synthetic binding compositions (such as antibody compositions). Without wishing to limit the invention to any theory or mechanism, it is believed that the technical feature of the present invention advantageously provides for synthetic binding compositions for multiplexing, that eliminate the necessity for multiple host species and concerns regarding cross-reactivity among related species, e.g., in indirect immunoassays. None of the presently known prior references or work has the unique inventive technical feature of the present invention.

Moreover, the prior references teach away from the present invention. For instance, conventional indirect immunoassays for multiplexing face limitations due to the species specificity of primary and secondary antibodies (or sdABs, etc.). However, not all antibodies/sdABs may be available or applicable for a given target across all species hosts required for multiplexing. Additionally, separate species-specific secondary antibodies that do not cross-react are necessary. For example, while most modern microscopes can achieve a 5-plex assay with ease (or higher), creating a panel of 5 different primary antibodies in different species (for the 5 targets of interest) and obtaining 5 distinct secondary antibodies for indirect detection is exceedingly challenging. Consequently, much of the research is confined to shallower multiplexes (e.g., 2-plex or 3-plex assays), or primary-labeled antibodies are utilized. However, these primary-labeled antibodies typically exhibit lower brightness and total DOLs (4-5) compared to using an indirect secondary system (16-25).

Another unique and inventive technical feature of the present invention is the use of amino acid optimization with substitutions, additions, or deletions with the synthetic binding composition (e.g., antibody compositions). Without wishing to limit the invention to any theory or mechanism, it is believed that the technical feature of the present invention advantageously provides for antibodies with a brighter signal and reduced noise. None of the presently known prior references or work has the unique inventive technical feature of the present invention.

Furthermore, the inventive technical features of the present invention contributed to a surprising result. For example, some antibody compositions described herein are two to three times brighter than currently available primary antibodies for direct immunofluorescence (see FIG. 8). Additionally, the Inventors determined that modest sequence optimization modifications (e.g., substitution modifications) lead to increased degrees of labeling (DOLs) while using less dye than standard methods. Alternatively, too many modifications or too high of DOL breaks the antibody composition's avidity to the intended target. Lastly, as described herein, some antibody compositions of the present invention do not require modifications; however, adding peptides to the C-terminals of either or both chains can greatly add to the final DOL while also requiring less dye to get there. Contrastingly, larger peptide additions work well for ELISA but may hinder their use in ICC/IF applications in cells/tissue where penetration is key for finding the target.

A further unique and inventive feature of the present invention is the development of novel methods for achieving high degrees of labeling (DOLs) per target of the primary antibody (see FIG. 9). This is accomplished by incorporating additional epitopes into the primary binder (e.g., antibody), which can be specifically recognized by novel types of secondary binders (e.g., secondary antibodies) carrying conjugations in various form factors. Alternatively, binder fragments (e.g., antibody fragments) can be added to the primary binder (e.g., primary antibody) to enable binding to proteins with matching epitopes, or pseudo-epitopes, that carry conjugations in multiple possible form factors.

These innovative systems not only enable high DOLs but also facilitate specifically engineered multiplexing by controlling the antibody fragments and matching epitopes used. This approach differs from conventional methods, which typically involve secondary antibodies binding to species-specific epitopes of a primary antibody, a process that can generate considerable noise in practice. By leveraging these inventive technical features, the present invention offers a more refined and precise method for achieving high DOLs and enhanced multiplexing capabilities in antibody-based applications.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows non-limiting examples of primary binding proteins to a target, including a generic antibody, scFV, sdAB/VHH/nanobody, and synthetic/engineered binders. Examples of types of fusions that could recombinantly added to a primary binder (in these examples of type A-Antibody) including, none, a polypeptide/protein, other binders (antibodies, scFVs, sdABs/VHHs or multiples thereof, or epitopes or pseudo-epitopes for secondary binders or multiples thereof. Lastly, secondary binders, which could be of any type of binder described in primary binders or any natural or engineered protein with an epitope or pseudo-epitope for binding a binder fused a binder binding the main target. Any of the above could be conjugated with a reporter, drug, or protein that is meant to be directed to the main target.

FIG. 2 shows a non-limiting example of antibody compositions of the present invention.

FIG. 3 shows a non-limiting example of antibody compositions of the present invention.

FIGS. 4A and 4B show non-limiting examples of antibody compositions described herein. FIG. 4B shows a multiplexing example with 4× sdAb Design (FIG. 4A far right): 3 Different primary antibodies with 3 different linked sdAb's and their corresponding epitope linked proteins carrying 3 different reporters.

FIG. 5 shows non-limiting examples of antibody binding compositions described herein.

FIG. 6 shows non-limiting examples of antibody compositions described herein. Antibody compositions described herein linked to 1 or more scFV or sdAB epitopes per Heavy and/or Light Chains. For examples: (Left) 2× sdAB Epitopes per chain, total bound by sdAb linked to Conjugated Protein or; (Right) 2× sdAB Epitopes per chain, total bound by sdAb linked to a second sdAB that is bound to a Conjugated Protein.

FIG. 7 shows non-limiting examples of antibody compositions described herein. An antibody linked to 1 or more scFV or sdAB epitopes per Heavy and or Light Chains. For examples: (Left) Conjugated sdAb or (Right) Conjugated 3× sdAb bound to Antibody with linked sdAb epitopes.

FIG. 8 shows that antibody compositions described herein are labeled in a way that they are >2× brighter than commercially primary/direct labeled antibodies alone or >˜4× brighter when 2 or more antibody compositions are combined as a ‘Poly-mAb’.

FIG. 9 shows non-limiting examples of antibody compositions described herein consisting of epitopes recombinantly added to the antibody for binding by conjugated products. Immunofluorescence microscopy of Antibody with 1 sdAb epitope per chain (A), or 3 sdAb epitopes per chain (B) bound by reporter conjugated sdAbs.

Aforementioned figures were created with BioRender.com.

### Terms

Unless otherwise explained, all technical and scientific terms used herein have the same meaning as commonly understood by one of ordinary skills in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, the disclosures of which are incorporated in their entirety herein by reference.

Although methods and materials similar or equivalent to those described herein can be used to practice or test the disclosed technology, suitable methods and materials are described below. The materials, methods, and examples are illustrative only and not intended to be limiting.

Binder or Target Binder: A polypeptide, a peptide, or a fragment thereof that binds a target, an epitope, a pseudo-epitope, or the like. In general, as used herein, binders may include antibodies or fragments thereof, including but not limited to a fragment antigen-binding region (Fab region), F(ab′)2, Fab′, Fv (Fragment Variable), single-domain antibody (sdAb) fragment or a single-chain variable fragment (scFv). Alternatively, as used herein, the binders may include synthetic binders (e.g., Aptamer, affibody, Designed Ankyrin Repeat Protein (DARPins), or molecularly imprinted polymer) or engineered binders.

Primary Target Binder or Primary Binder: A polypeptide, a peptide, or a fragment thereof that directly and specifically binds to a target. As used herein, primary target binders may include antibodies or fragments thereof, including but not limited to a fragment antigen-binding region (Fab region), F(ab′)2, Fab′, Fv (Fragment Variable), single-domain antibody (sdAb) fragment or a single-chain variable fragment (scFv). Alternatively, as used herein, the primary target binders may include synthetic binders (e.g., Aptamer, affibody, Designed Ankyrin Repeat Protein (DARPins), or molecularly imprinted polymer) or engineered binders.

Secondary Target Binder or Secondary Binder: A polypeptide, a peptide, or a fragment thereof that indirectly binds to a target, either via directly binding to the primary target binder or being recombinantly linked to the primary target binder. As used herein, secondary target binders may include antibodies or fragments thereof, including but not limited to a fragment antigen-binding region (Fab region), F(ab′)2, Fab′, Fv (Fragment Variable), single-domain antibody (sdAb) fragment or a single-chain variable fragment (scFv). Alternatively, as used herein, the secondary target binders may include synthetic binders (e.g., Aptamer, affibody, Designed Ankyrin Repeat Protein (DARPins), or molecularly imprinted polymer) or engineered binders.

The present invention is not limited to primary target binders and secondary binders. In addition to these, the present invention may also encompass tertiary binders, quaternary binders, quinary binders, senary binders, etc. For instance, a tertiary binder, akin to a secondary binder, may comprise a polypeptide, a peptide, or a fragment thereof that indirectly binds to a target, either by directly binding to the secondary target binder or through recombinant linkage thereto. Likewise, a quaternary binder may comprise a polypeptide, a peptide, or a fragment thereof that indirectly binds to a target, either by directly binding to the tertiary target binder or through recombinant linkage thereto, and so forth. Furthermore, each binder may additionally include a conjugated reporter molecule (or drug molecule). For example, the reporter (or drug molecule) may be conjugated to at least one conjugation-targeted amino acid residue in the binder. Alternatively, in some embodiments, the reporter (or drug molecule) may be conjugated to at least one post-translational modification on the binder.

Antibody (AB): A polypeptide that includes at least a light chain and/or heavy chain immunoglobulin variable region and specifically binds an epitope of an antigen. Heavy chain antibodies, such as those found in sharks and camelids (the precursors for nanobodies), are also included in this definition. Most commonly, AB refers to primary recombinant monoclonal antibodies or mAbs. Antibodies include monoclonal antibodies, polyclonal antibodies, or fragments of antibodies. Merely by way of example, monoclonal antibodies can be prepared from murine hybridomas according to classical methods such as Kohler and Milstein (Nature 256:495-97, 1975) or derivative methods thereof. Detailed procedures for monoclonal antibody production are described, for example, by Harlow and Lane, Using Antibodies: A Laboratory Manual, CSHL, New York, 1999. An antibody can be conjugated or otherwise labeled with a detectable label, such as a fluorophore (e.g., a reporter).

As used herein, an “antibody fragment” refers to a partial or truncated version of an antibody that retains the ability to specifically bind to its target antigen. Non-limiting examples of antibody fragments include, but are not limited to, Fab, F(ab′)2, Fab′, Fv, scFv, sdAb, VHH, and dAb fragments.

In addition to antibody fragments, synthetic binders that are not derived from antibodies can also be used in the present invention. These synthetic binders are designed to specifically bind target antigens and may include, but are not limited to, aptamers, affibodies, DARPins (Designed Ankyrin Repeat Proteins), and molecularly imprinted polymers. The use of these synthetic binders can provide alternative strategies for targeting and binding specific antigens, potentially expanding the versatility and applicability of the invention.

Fragment Antigen-Binding Region (Fab region): a region on an antibody that binds to antigens. The Fab region may comprise one constant and one variable domain of each of the heavy and the light chain. As used herein, “fragment antigen-binding region” and “variable region” may be used interchangeably.

F(ab′)2: A bivalent antibody fragment that consists of two Fab regions joined together by disulfide bonds. F(ab′)2 retains the ability to bind two antigens simultaneously while lacking the Fc region of a full antibody.

Fab′: A monovalent antibody fragment that consists of one variable domain and one constant domain of the heavy chain, and one variable domain of the light chain, with an intact hinge region. Fab′ retains the ability to bind antigens and can be further conjugated or linked to other molecules through the hinge region.

Fv: Fragment Variable, the smallest functional unit of an antibody that is capable of binding antigens. It consists of the variable domains of the heavy (VH) and light (VL) chains.

Single-Chain Variable Fragment (scFv): a fusion protein of the Fab region wherein the heavy and light chains are synthesized as a singular protein instead of two chains.

Fragment crystallizable region (Fc region): a backbone region on an antibody that does not interact with antigens. The Fc may comprise two heavy chains. As used herein, “fragment crystallizable region” and “constant region” may be used interchangeably.

Single Domain Antibodies (sdAb): an antibody fragment consisting of a single monomeric variable antibody domain. As used herein, “nanobody (NB),” “VHH,” “sdAb,” or “dAb” may be used interchangeably.

VHH: Variable domain of the Heavy chain of a Heavy-chain antibody, a type of sdAb derived from camelids. VHH fragments are also known as “nanobodies” or “dAbs” and retain the ability to bind antigens with high specificity and affinity while being smaller and more stable than conventional antibody fragments.

dAb: Domain Antibody, another term for sdAb, referring to a single monomeric variable antibody domain derived from heavy-chain-only antibodies. As used herein, “nanobody (NB),” “VHH,” “sdAb,” or “dAb” may be used interchangeably.

Aptamers: Short, single-stranded nucleic acid molecules (DNA or RNA) that can fold into unique three-dimensional structures and bind to specific target molecules with high affinity and specificity. Aptamers are selected through a process called SELEX (Systematic Evolution of Ligands by Exponential enrichment) and can be used as alternatives to antibodies in various applications, such as diagnostics, therapeutics, and research.

Affibodies: Small, engineered, non-immunoglobulin-based scaffold proteins derived from the Z domain of staphylococcal protein A. Affibodies can be engineered to bind a wide range of target molecules with high affinity and specificity. Due to their small size, high stability, and ease of production, they are suitable alternatives to antibodies for various applications, including diagnostics, therapeutics, and imaging.

DARPins (Designed Ankyrin Repeat Proteins): Small, highly stable, and versatile engineered scaffold proteins composed of multiple ankyrin repeat domains. DARPins can be designed to bind target molecules with high affinity and specificity. They offer advantages over traditional antibodies in terms of size, stability, solubility, and ease of production, making them suitable for various applications, including diagnostics, therapeutics, and research.

Molecularly Imprinted Polymers (MIPs): Synthetic polymers that possess specific recognition sites for target molecules. MIPs are created by polymerizing monomers in the presence of a template molecule, which is later removed to leave behind binding cavities that are complementary in shape, size, and functional group orientation to the template. MIPs can selectively bind to their target molecules with high affinity, and their robustness and stability make them attractive alternatives to antibodies for applications such as sensing, separation, and catalysis.

Engineered Binding Proteins (EBPs) or Artificial Binding Proteins (ABPs): A diverse class of synthetic or computationally designed proteins that can specifically recognize and bind to target molecules with high affinity and specificity. These binding proteins can be developed using various techniques, including rational design, directed evolution, deep learning, and artificial intelligence-driven approaches. EBPs or ABPs offer a versatile alternative to traditional antibodies and other natural binding proteins for various applications in diagnostics, therapeutics, and research.

“Polypeptide” and “Protein” may be used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

Peptide: a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

Epitope: a region that is recognized and bound to by immunological binders, Antibodies, scFVs, sdABs (nanobodies), etc, from immune derived constructs.

Pseudo-epitope: a region that is recognized and bound to by non-immuno constructs, such as EBPs, ABPs.

Degree of Labeling (DOL): refers to how many fluorophores/drugs/other molecules are conjugated to an AB/NB/AB-Fragment/Protein.

Primary Antibody: an antibody that directly binds the target of interest.

Secondary Antibody: an antibody(s) that binds the primary AB to amplify a signal.

Multiplex,-ed,-ing: Embodiments of the present invention allow multiple targets in a sample to be detected substantially simultaneously or sequentially, as desired, using multiple different conjugates. Multiplexing can include identifying and/or quantifying nucleic acids generally, DNA, RNA, peptides, proteins, both individually and in any and all combinations. Multiplexing also can include detecting two or more of a gene, a messenger, and a protein in a cell in its anatomic context.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

### Binder Compositions Overview

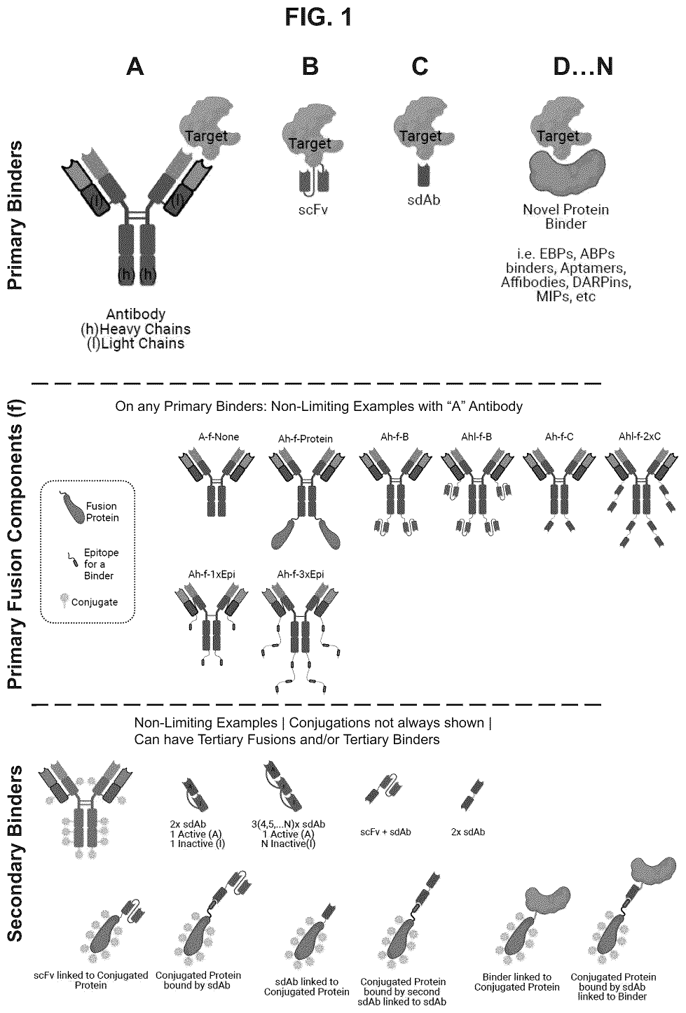

Referring to FIG. 1, the present invention may feature an engineered binder composition. In some embodiments, the engineered binder composition as described herein may comprise a primary target binder configured to directly bind a target and a reporter (or drug molecule) conjugated to at least one conjugation-targeted amino acid residue in the primary target binder or to a post-translational modification such as a glycosylation.

Alternatively, the present invention may feature a synthetic binder composition comprising a) a primary target binder configured to directly bind a target molecule, b) a primary fusion component attached (e.g., at either a C or N terminal) to the primary target binder, and c) a reporter (or drug molecule) conjugated to either (i) at least one conjugation-targeted amino acid residue in either one or both of (i) the primary target binder or (ii) the primary fusion component or (ii) to at least one post-translational modification on one or both of (i) the primary target binder or (ii) the primary fusion component. In some embodiments, the primary fusion component is recombinantly linked to the primary target binder. In other embodiments, the primary fusion component binds directly to the primary target binder. The primary fusion composition may either be (i) a binding component that allows the binding of a secondary binder to the primary target binder (e.g., an epitope or a pseudo-epitope) or (ii) a secondary binder. In certain embodiments featuring the primary fusion component as a secondary binder, the secondary binder may either be recombinantly linked to the primary target binder or directly bind to it.

Alternatively, in some embodiments, the compositions described herein may comprise binder fragments (e.g., antibody fragments) that can be added to the primary binder (e.g., primary antibody) to enable binding to proteins with matching epitopes that carry conjugations in multiple possible form factors. For example, the protein, whether natural or engineered, that lacks a binder (e.g., a binder conjugated thereto), a secondary target, epitope, or pseudo-epitope may be attached. This enables the protein to bind to a primary/secondary binder or other xxx-ary binder. Specifically, as illustrated in FIG. 3, a protein is depicted with an epitope fused to it. In some embodiments, the epitope is not fused to the binder; rather, the secondary binder may bind to a natural epitope, or pseudo-epitope, present on the protein. Additionally, reference is made to the conjugated version portrayed in FIG. 3B.

In certain embodiments, the described composition may additionally include a protein bearing solely an epitope or pseudo-epitope, serving the purpose of transporting labels to the primary or secondary binders.

In some embodiments, the conjugation-targeted amino acid residues (e.g., lysine, cysteine, or others, both naturally occurring and substituted) throughout the synthetic binder composition (e.g., throughout the primary binder composition, the primary fusion component, the secondary binder, etc.) are spaced such that when the reporter (or drug molecule) is conjugated to the conjugation-targeted amino acid residue one reporter (or drug molecule) does not interfere with the conjugation of another reporter (or drug molecule). In some embodiments, the conjugation-targeted amino acid residues (e.g., lysine, cysteine, or others, both naturally occurring and substituted) throughout the antibody are spaced to optimize the performance of the conjugated molecule, with no, minimal, or enhanced binding affinity changes. This optimal spacing may vary but could fall within a 3D space range of 10 Å to 100 Å, depending on the specific amino acid, type of conjugated molecule, and conjugation chemistry employed.

Embodiments are not limited to specific substitutions. In some embodiments, the compositions described herein are further modified (e.g., substitution, deletion, or addition of standard amino acids; chemical modification, etc.). Modifications that are understood in the field include N-terminal modification, C-terminal modification (which protects the peptide from proteolytic degradation), alkylation of amide groups, hydrocarbon “stapling” (e.g., to stabilize alpha-helix conformations). In some embodiments, the peptides described herein may be modified by conservative residue substitutions, for example, of the charged residues (K to R, R to K, D to E, and E to D). In some embodiments, such conservative substitutions provide subtle changes, for example, to the receptor binding sites with the goal of improving specificity and/or biological activity. Modifications of the terminal carboxy group include, without limitation, the amide, lower alkyl amide, constrained alkyls (e.g., branched, cyclic, fused, adamantyl) alkyl, dialkyl amide, and lower alkyl ester modifications.

Without wishing to limit the present invention to any theory or mechanism, it is believed that selectively inserting, substituting, or deleting specific amino acids allows for optimized amino acid conjugation (e.g., optimized amino acid spacing) throughout the composition.

For example, in some embodiments, the conjugation-targeted amino acid residues (e.g., naturally occurring and substituted) throughout the synthetic binder composition are spaced about 25 Angstroms (Å) apart. In some embodiments, the conjugation-targeted amino acid residues (e.g., naturally occurring and substituted) throughout the synthetic binder composition are spaced about 50 Å apart. In some embodiments, the conjugation-targeted amino acid residues (e.g., naturally occurring and substituted) throughout the synthetic binder composition are spaced about 75 Å apart. In some embodiments, the conjugation-targeted amino acid residues (e.g., naturally occurring and substituted) throughout the synthetic binder composition are spaced about 100 Å apart. In some embodiments, the conjugation-targeted amino acid residues (e.g., naturally occurring and substituted) throughout the synthetic binder composition are spaced about 150 Å apart.

Without wishing to limit the present invention to any theory or mechanism, it is believed that appropriate spacing between conjugated molecules is important to minimize quenching or interference. This spacing helps ensure optimal signal generation or drug delivery. Furthermore, conjugation may be optimized using an increasing gradient of Label/Conjugate to Binder molar ratios. Non-limiting examples of ratios (e.g., Dye:Antibody) that may be examined include 5:1, 10:1, 15:1, 25:1, 35:1, 50:1, and 100:1. Subsequently, all conjugated species may further undergo testing for their functionality, either through Surface Plasmon Resonance (SPR) or via their intended applications such as Immunofluorescence (IF), Immunohistochemistry (IHC), Immunocytochemistry (ICC), Enzyme-Linked Immunosorbent Assay (ELISA), and others.

In certain embodiments, the conjugation-targeted amino acid residues may be linked with a reporter molecule. Alternatively, in other embodiments, these residues could be conjugated with a drug molecule. Further variations include the conjugation of post-translational modifications such as a glycosylation moiety. Additionally, some embodiments may feature a combination of conjugation types throughout the described compositions; for example, a composition may include both a reporter and a drug.

In some embodiments, the primary target binder comprises an antibody or a fragment thereof. Non-limiting examples of an antibody fragment used herein may include a fragment antigen-binding region (Fab region), F(ab′)2, Fab′, Fv (Fragment Variable), single-domain antibody (sdAb) fragment, or a single-chain variable fragment (scFv). In other embodiments, the primary target binder comprises synthetic binders or engineered binders. Non-limiting examples of a synthetic binder may include an aptamer, affibody, Designed Ankyrin Repeat Protein (DARPins), or molecularly imprinted polymer.

Regarding the primary fusion component, in some embodiments, the primary fusion component is recombinantly linked to the C terminal of the primary target binder. In other embodiments, the primary fusion component is recombinantly linked to the N terminal of the primary target binder. Alternatively, in certain embodiments, the primary fusion component is recombinantly linked to an internal portion of the primary target binder. Furthermore, in certain embodiments, the primary fusion component may directly bind to the primary target binder.

Examples of primary fusion components may include but are not limited to proteins, epitopes, pseudo-epitopes, or a combination thereof.

In certain embodiments, the primary fusion component is a binding element (such as an epitope or pseudo-epitope), facilitating the attachment of a secondary binder to the primary target binder. In such embodiments, when the primary fusion component functions as a binding element, the primary fusion component is recombinantly linked to the primary target binder. In other embodiments, the primary fusion component is a secondary binder. In such embodiments, in which the primary fusion component is a secondary binder, the secondary binder may either be recombinantly linked to the primary target binder or directly bind to it.

In some embodiments, the synthetic binder compositions described herein may comprise two or more primary fusion components, wherein the two or more primary fusion components are recombinantly linked to the primary target binder in tandem. In some embodiments, the synthetic binder compositions described herein may comprise three or more primary fusion components, wherein the three or more primary fusion components are recombinantly linked to the primary target binder in tandem. In some embodiments, the synthetic binder compositions described herein may comprise four or more primary fusion components, wherein the four or more primary fusion components are recombinantly linked to the primary target binder in tandem. In some embodiments, the synthetic binder compositions described herein may comprise five or more primary fusion components, wherein the five or more primary fusion components are recombinantly linked to the primary target binder in tandem. In some embodiments, the synthetic binder compositions described herein may comprise ten or more primary fusion components, wherein the ten or more primary fusion components are recombinantly linked to the primary target binder in tandem.

In some embodiments, the synthetic binder composition further comprises a secondary binder. In some embodiments, the secondary binder is recombinantly linked to the primary target binder. In other embodiments, the secondary binder binds directly to the primary target binder.

The secondary binder may comprise an antibody or a fragment thereof, such as a fragment antigen-binding region (Fab region), F(ab′)2, Fab′, Fv (Fragment Variable), single-domain antibody (sdAb) fragment, or a single-chain variable fragment (scFv). In other embodiments, the secondary binder may comprise synthetic binders (e.g., aptamers, affibodies, Designed Ankyrin Repeat Protein (DARPins), or molecularly imprinted polymers) or engineered binders.

In some embodiments, the secondary binder further comprises a reporter (or drug molecule) conjugated to at least one conjugation-targeted amino acid residue in the secondary binder. According to the previously stated, in some embodiments, the conjugation-targeted amino acid residues (e.g., lysine, cysteine, or others, both naturally occurring and substituted) throughout the secondary binder are spaced such that when the reporter (or drug molecule) is conjugated to the conjugation-targeted amino acid residue one reporter (or drug molecule) does not interfere with the conjugation of another reporter. In further embodiments, the secondary target binder may further comprise a reporter or drug conjugated to at least one post-translational modification in the secondary target binder.

In some embodiments, the secondary binder further comprises a secondary fusion component. In some embodiments, the secondary fusion component is recombinantly linked to the C terminal of the secondary target binder. In other embodiments, the secondary fusion component is recombinantly linked to the N terminal of the secondary target binder. Alternatively, in certain embodiments, the secondary fusion component is recombinantly linked to an internal portion of the secondary target binder. Furthermore, in certain embodiments, the secondary fusion component may directly bind to the secondary target binder.

In certain embodiments, the secondary fusion component is a binding element (such as an epitope or pseudo-epitope), facilitating the attachment of a tertiary binder to the secondary target binder. In such embodiments, when the secondary fusion component functions as a binding element, the secondary fusion component is recombinantly linked to the secondary target binder. In other embodiments, the secondary fusion component is a tertiary binder. In such embodiments, in which the secondary fusion component is a tertiary binder, the tertiary binder may either be recombinantly linked to the secondary binder or directly bind to it. In further embodiments, the secondary fusion component may further comprise a conjugated post-translational modification in the secondary fusion component.

The synthetic binder compositions as described herein are not limited to the aforementioned binders (e.g., primary target binders and secondary binders). In addition to these, the synthetic binder composition may also include other binders, e.g., tertiary binders, quaternary binders, quinary binders, senary binders, etc. For instance, a tertiary binder, akin to a secondary binder, may comprise a polypeptide, a peptide, or a fragment thereof that indirectly binds to a target, either by directly binding to the secondary target binder or through recombinant linkage thereto. Likewise, a quaternary binder may comprise a polypeptide, a peptide, or a fragment thereof that indirectly binds to a target, either by directly binding to the tertiary target binder or through recombinant linkage thereto, and so forth. Furthermore, each binder may additionally include a reporter molecule (or drug molecule) conjugated thereto (e.g., either to at least one conjugation-targeted amino acid residue therein or to at least one post-translational modification thereon).

In some embodiments, the reporter is a fluorescence reporter, fluorochrome, or enzyme reporter. Non-limiting examples of an enzyme reporter include a chromogen such as 3,3′-diaminobenzidine tetrahydrochloride (DAB), horseradish peroxidase (HRP), or alkaline phosphatase (AP). In some embodiments, the fluorescence reporter is a fluorescence dye. In other embodiments, the reporter is an oligonucleotide. In certain embodiments, the reporter is a metal isotope.

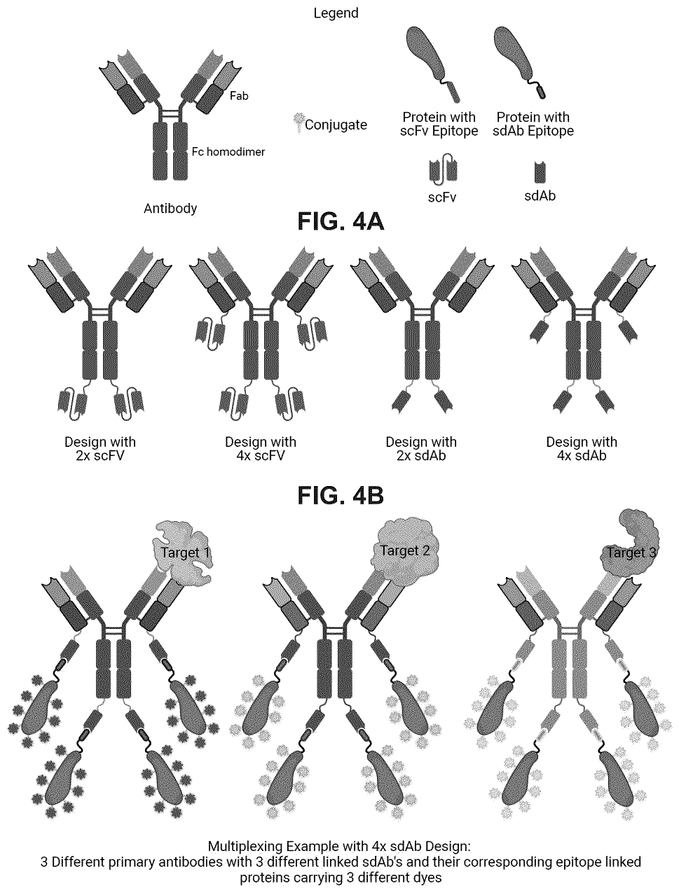

In certain embodiments, the aforementioned compositions may be utilized individually against a single target. Alternatively, in other embodiments, they may be combined with each other to target multiple entities for multiplexing purposes (e.g., as depicted in FIG. 4B). Alternatively, a hybrid approach combining both methods may also be employed.

The present invention may additionally include kits comprising the synthetic binder compositions described herein, either individually or in combination with other materials. For instance, kits might pair the compositions with off-the-shelf materials, such as a DNA dye marker, for enhanced utility.

The present invention further extends to methods for employing the compositions disclosed herein. These methods encompass a broad array of applications, including diagnostics, research, and therapeutic interventions. Diagnostic applications involve detecting and quantifying specific targets, while research applications encompass studying protein interactions and cellular pathways. Therapeutically, the compositions may facilitate targeted drug delivery or immunotherapy. Methods may include immunoassays, microscopy, flow cytometry, and drug screening assays.

### Non-limiting Examples of Binder Compositions

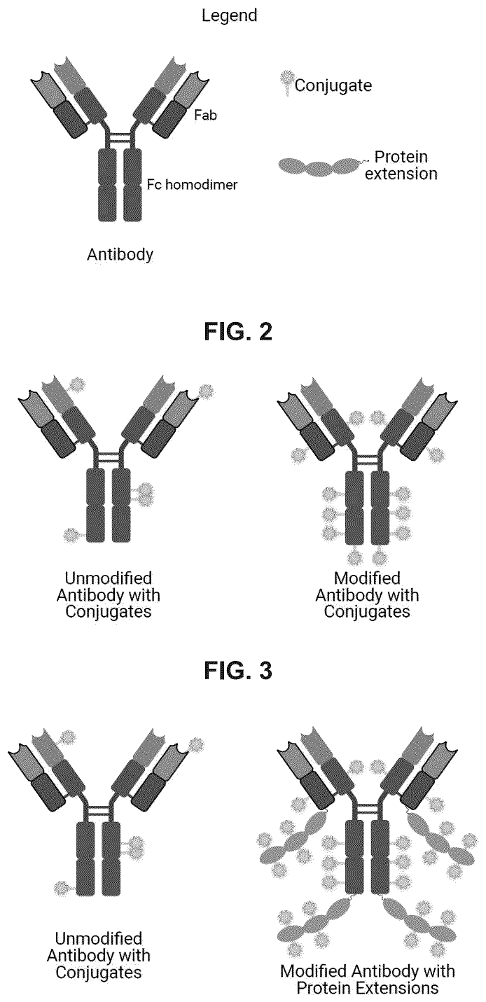

Referring now to FIG. 2, the present invention may feature an isolated antibody composition. The isolated antibody composition may comprise a constant region (Fc) comprising a heavy chain and a variable region (Fab) comprising a light chain and a portion of a heavy chain. In some embodiments, the constant region comprises one or more amino acid substitutions. At least one of the one or more amino acid substitutions comprises an amino acid substituted to a lysine residue or a cysteine residue. In some embodiments, a reporter is conjugated to at least one of the lysine or cysteine residues. In other embodiments, a drug molecule is conjugated to at least one of the lysine or cysteine residues. Naturally occurring and substituted lysine or cysteine residues throughout the constant region may be spaced such that when the reporter (or drug molecule) is conjugated to a lysine or cysteine residue, one reporter (or drug molecule) does not interfere with the conjugation of another reporter (or drug molecule). In other embodiments, the one of the one or more amino acid substitutions in the constant region comprises a naturally occurring lysine or cysteine residue substituted to a conserved amino acid residue. In some embodiments, the conserved amino acid residue allows for complementary function but no conjugation of a reporter (or drug molecule).

The present invention also features an isolated antibody composition comprising a constant region (Fc) and a variable region (Fab). The constant region may comprise one or more amino acid substitutions, wherein at least one of the one or more amino acid substitutions comprises an amino acid substituted to a lysine or cysteine residue. In some embodiments, the variable region comprises one or more amino acid substitutions, wherein at least one of the one or more amino acid substitutions comprises an amino acid substituted to a lysine or cysteine residue. In some embodiments, a conjugation occurs at at least one of the lysine or cysteine residues. In some embodiments, naturally occurring and substituted lysine or cysteine residues throughout the constant region and/or variable region are spaced such that when there is a conjugation to a lysine or cysteine residue, it has limited interference with conjugations on another lysine or cysteine residue. In some embodiments, one of the one or more amino acid substitutions in the constant region or variable region comprises a naturally occurring lysine or cysteine residue substituted to a conserved amino acid residue, wherein the conserved amino acid residue allows for complementary function but no conjugation.

In alternative embodiments, the antibody compositions described herein do not comprise one or more amino acid substitutions and may further comprise the undermentioned embodiments.

In some embodiments, the aforementioned compositions may further comprise an additional polypeptide sequence with exposed lysines or cysteines for conjugation added to a C-terminus of a heavy chain, an additional polypeptide sequence with exposed lysines or cysteines for conjugation added to a C-terminus of a light chain, or a combination thereof.

Additionally, the aforementioned compositions, in some embodiments, may further comprise an additional polypeptide sequence of one or more AB-fragments such as single-domain antibody (sdAb) fragment or single-chain variable fragment(s) (scFv) added to a C-terminus of a heavy chain, an additional polypeptide sequence of one or more scFv or sdAB added to a C-terminus of a light chain, or a combination thereof. In some embodiments, the additional polypeptide sequence is able to bind to a conjugated protein with the corresponding epitope(s) for the said additional polypeptide sequence of the scFv or sdAB.

In some embodiments, the aforementioned compositions may further comprise an additional polypeptide sequence with one or more epitopes to a C-terminus of a heavy chain, an additional polypeptide sequence with one or more epitopes to a C-terminus of a light chain, or a combination thereof. In some embodiments, the additional polypeptide sequence is able to be bound to by a corresponding single-domain antibody (sdAb) fragment, a single-chain variable fragment (scFv), or an antibody (AB).

The present invention may further feature a single-domain antibody (sdAb) fragment or a single-chain variable fragment (scFv); and at least one of: a polypeptide sequence or a polypeptide sequence of one or more additional scFv or sdAB. In some embodiments, the scFV or sdAB are conjugated or bound to a conjugated protein with the corresponding epitope(s) for the added scFv or sdAb.

Modifications (e.g., amino acid substitutions) to the constant region of the antibody composition described herein may be determined by looking at known crystal structures. Subsequently, after modification, the antibody compositions are screened (e.g., using machine learning/artificial intelligence models or Deepmind's AlphaFold 2) to determine if the new structure is stable or not.

Embodiments are not limited to specific substitutions. In some embodiments, the antibody compositions described herein are further modified (e.g., substitution, deletion, or addition of standard amino acids; chemical modification, etc.). Modifications that are understood in the field include N-terminal modification, C-terminal modification (which protects the peptide from proteolytic degradation), alkylation of amide groups, hydrocarbon “stapling” (e.g., to stabilize alpha-helix conformations). In some embodiments, the peptides described herein may be modified by conservative residue substitutions, for example, of the charged residues (K to R, R to K, D to E, and E to D). In some embodiments, such conservative substitutions provide subtle changes, for example, to the receptor binding sites with the goal of improving specificity and/or biological activity. Modifications of the terminal carboxy group include, without limitation, the amide, lower alkyl amide, constrained alkyls (e.g., branched, cyclic, fused, adamantyl) alkyl, dialkyl amide, and lower alkyl ester modifications.

In some embodiments, the antibody compositions described herein are made recombinantly using synthesized oligos.

In some embodiments, the lysine or cysteine residues (e.g., naturally occurring and substituted) throughout the antibody composition (e.g., the constant region (Fc)) are spaced about 25 Angstroms (Å) apart. In some embodiments, the lysine or cysteine residues throughout the antibody composition (e.g., the constant region (Fc)) are spaced about 50 Å apart. In some embodiments, the lysine or cysteine residues throughout the antibody composition (e.g., the constant region (Fc)) are spaced about 75 Å apart. In some embodiments, the lysine or cysteine residues throughout the antibody composition (e.g., the constant region (Fc)) are spaced about 100 Å apart. In some embodiments, the lysine or cysteine residues throughout the antibody composition (e.g., the constant region (Fc)) are spaced about 150 Å apart.

In some embodiments, the lysine or cysteine residues (e.g., naturally occurring and substituted) are spaced such that there is at least 25 Å between two reporters (e.g., between the center of the two reporters). In some embodiments, the lysine or cysteine residues are spaced such that there is at least 50 Å between two reporters. In some embodiments, the lysine or cysteine residues are spaced such that there is at least 75 Å between two reporters. In some embodiments, the lysine or cysteine residues are spaced such that there is at least 100 Å between two reporters. In some embodiments, the lysine or cysteine residues are spaced such that there is at least 150 Å between two reporters.

Without wishing to limit the present invention to any theory or mechanism, it is believed that spacing between the reporter at least 25 Å apart limits the quenching of the reporter.

A conservative substitution, as known to one of ordinary skills in the art, refers to a complete replacement of an amino acid residue with a different residue having similar biochemical characteristics, such as size, charge, polarity, etc. For instance, the aromatic Tyrosine may be conservatively substituted with aromatic phenylalanine, or basic Arginine may be conservatively substituted with basic Lysine. Tables 1A and 1B show non-limiting examples of conservative amino acid substitutions.

Table 1A and 1B:

TABLE 1A

| Original Residue | Conservative Substitutions |
|---|---|
| Ala (A) | Cys, Gly, Ser, Thr, Val |
| Arg (R) | Asn, Gln, Glu, His, Lys |
| Asn (N) | Arg, Asp, Gln, Glu, His, Lys, Ser, Thr |
| Asp (D) | Asn, Gln, Glu, Ser |
| Cys (C) | Ala, Ser |
| Gln (Q) | Arg, Asn, Asp, Glu, His, Lys, Met, Ser |
| Glu (E) | Arg, Asn, Asp, Gln, His, Lys, Ser |
| Gly (G) | Ala, Ser, Glu, Asp |
| Ile (I) | Leu, Met, Phe, Val |
| Leu (L) | Ile, Met, Phe, Val |
| Lys (K) | Arg, Asn, Gln, Glu, Ser |
| Met (M) | Gln, Ile, Leu, Phe, Val |
| Phe (F) | Ile, Leu, Met, Trp, Tyr |
| Pro (P) | None |
| Ser (S) | Ala, Asn, Asp, Gln, Glu, Gly, Lys, Thr |
| Thr (T) | Ala, Asn, Ser, Val |
| Trp (W) | Phe, Tyr |
| Tyr (Y) | His, Phe, Trp, |
| Val (V) | Ala, Ile, Leu, Met, Thr |

TABLE 1B

| Amino Acid Property | Amino Acid Substitutions |
|---|---|
| Hydrophobic | Cys, Ile, Leu, Met, Phe, Pro, Trp, Val |
| Aliphatic | Ala, Ile, Leu, Pro, Val |
| Aromatic | His, Phe, Trp, Tyr |
| Amide | Asn, Gln |
| Nucleophilic | Cys, Ser, Thr |
| Polar | Arg, Asp, Asn, Gln, Glu, Lys |
| Negative | Asp, Glu |
| Positive | Arg, Lys, His |
| Small | Ala, Gly, Pro, Ser |
| C-beta | Ile, Thr, Val |

The isolated antibody compositions described herein may further comprise a protein or a peptide thereof linked to the constant region (see FIG. 3, right). In some embodiments, a reporter (e.g., a fluorescence reporter or an enzymatic reporter) or a drug molecule may be conjugated to the protein or peptide thereof. In some embodiments, a reporter or drug molecule is conjugated to at least one residue of the protein. In some embodiments, the reporter or drug molecule is conjugated to at least one lysine residue of the protein. In other embodiments, the reporter or drug molecule is conjugated to at least one cysteine residue in the protein.

In some embodiments, the protein (and/or peptide thereof) may be directly linked to the constant region. In other embodiments, the protein (and/or peptide thereof) may be indirectly linked to the constant region. In some embodiment, the protein or the peptide thereof further comprises an epitope sequence for binding to a single-domain antibody (sdAb) fragment or a single-chain variable fragment (scFv).

Without wishing to limit the present invention to any theory or mechanism, it is believed that the proteins and/or peptides used herein are small enough (e.g., less than 300 kDa) to ensure diffusion into a cell or tissue to a target. Additionally, the proteins (and/or peptides thereof) used herein may be screened to determine if the naturally occurring lysines and cysteines within the sequence are properly spaced to allow for optimal conjugation of a reporter as aforedescribed.

In some embodiments, the proteins and/or peptides thereof will only bind to a target and will not bind or be bound by anything else in the cells or tissues. In some embodiments, the proteins used herein are naturally occurring, e.g., known proteins. In other embodiments, the proteins used herein are human or computer-generated proteins, e.g., synthetic proteins.

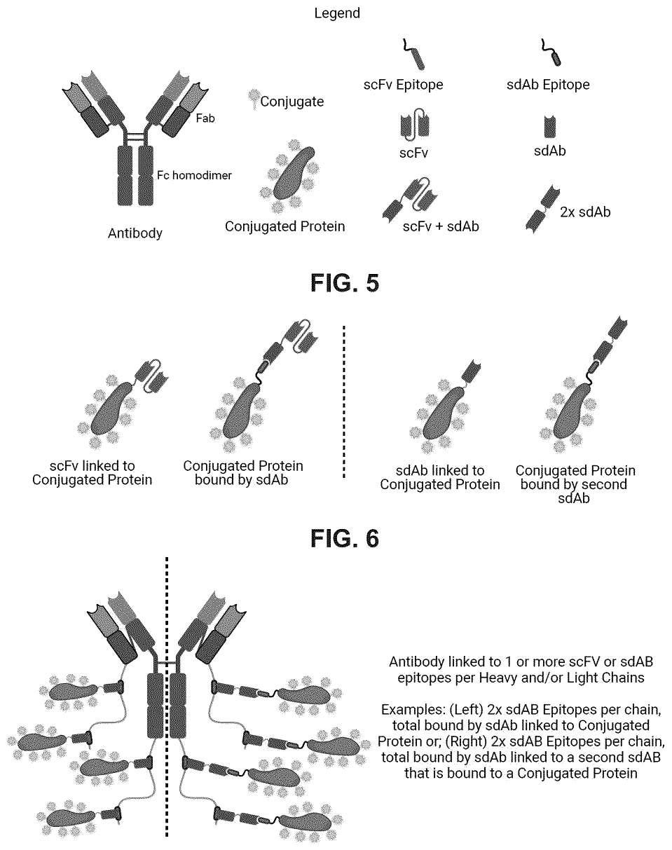

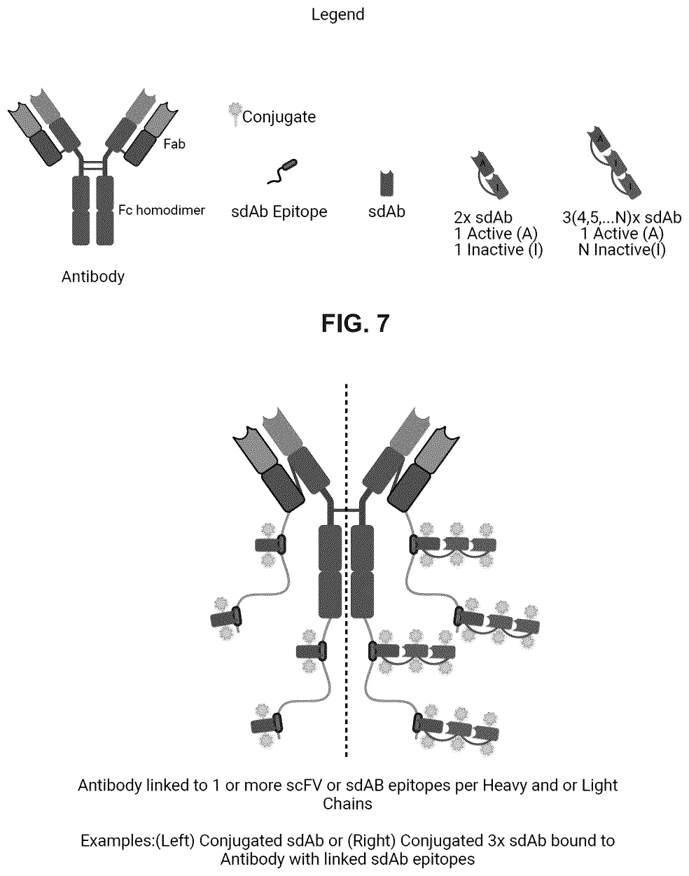

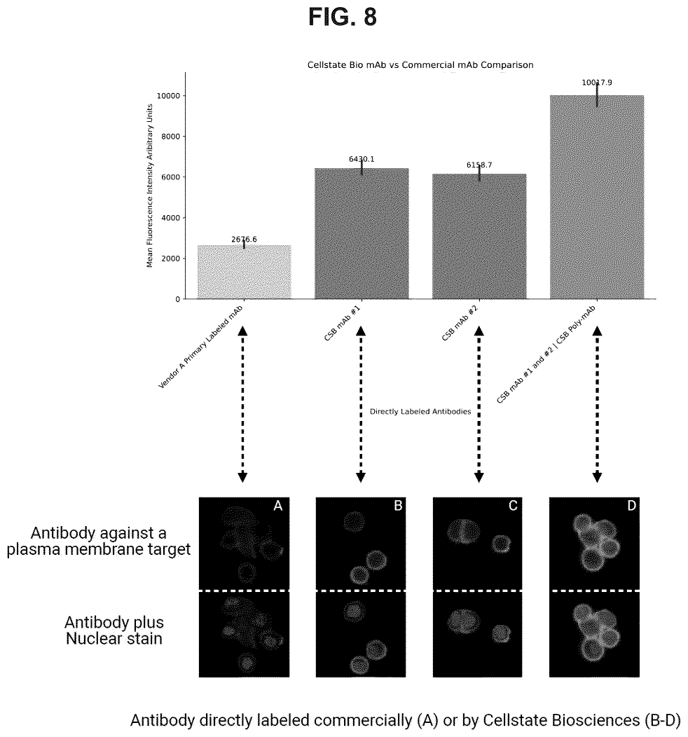

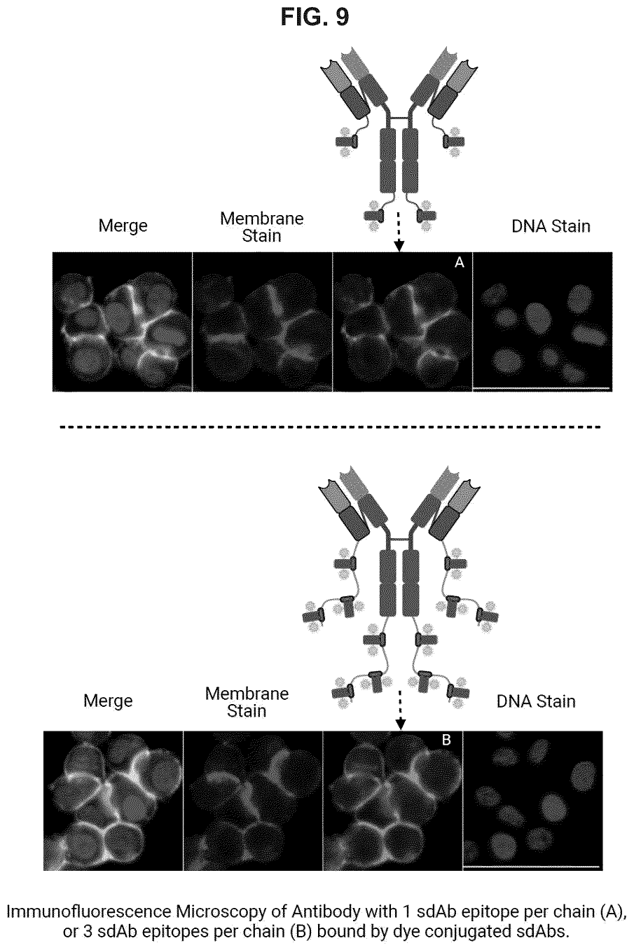

Now referring to FIG. 5, the present invention features an isolated antibody composition comprising at least one variable region (Fab) comprising a light chain and a portion of a heavy chain (e.g., a full-length heavy chain or a portion thereof), and a protein linked to the portion of the heavy chain. The protein may comprise one or more amino acid substitutions, wherein at least one of the one or more amino acid substitutions comprises an amino acid substituted to a lysine or cysteine residue. In some embodiments, a reporter is conjugated to at least one of the lysine or cysteine residues. In other embodiments, a drug molecule is conjugated to at least one of the lysine or cysteine residues. Naturally occurring and substituted lysine or cysteine residues throughout the constant region may be spaced such that when the reporter (or drug molecule) is conjugated to a lysine or cysteine residue, one reporter (or drug molecule) does not interfere with the conjugation of another reporter (or drug molecule). In some embodiments, one of the one or more amino acid substitutions in the protein comprises a naturally occurring lysine or cysteine residue substituted to a conserved amino acid residue, wherein the conserved amino acid residue allows for complementary function but no conjugation of a reporter or a drug molecule.

Additionally, the proximity of tryptophan (Trp), tyrosine (Tyr), methionine (Met), and histidine (His) residues may be spaced throughout the antibody compositions described herein. For example, the aforementioned residues may be spaced such that said residues are not within three amino acids of a cysteine or lysine residue. Without wishing to limit the present invention to any theories or mechanisms, it is believed that the proximity of a Trp, Tyr, Met, or His residue to a Lys or Cys residue can quench a reporter (e.g., a fluorescence reporter).

In some embodiments, the protein comprises one or more amino acid substitutions. At least one of the one or more amino acid substitutions comprises an amino acid substituted to a lysine or cysteine residue. In some embodiments, a reporter is conjugated to at least one of the lysine or cysteine residues. In other embodiments, a drug molecule is conjugated to at least one of the lysine or cysteine residues. In some embodiments, naturally occurring and substituted lysine or cysteine residues throughout the protein are spaced such that when a reporter (or drug molecule) is conjugated to a lysine or cysteine residue, one reporter (or drug molecule) does not interfere with the conjugation of another reporter (or drug molecule). In other embodiments, the one of the one or more amino acid substitutions in the protein comprises a naturally occurring lysine or cysteine residue substituted to a conserved amino acid residue. In some embodiments, the conserved amino acid residue allows for complementary function but no conjugation of a reporter or drug molecule.

The proteins or peptides thereof described herein may be made recombinantly by design. In some embodiments, the proteins or peptides thereof are not made by mutagenesis.

The present invention features an isolated antibody composition comprising at least one variable region (Fab) comprising a light chain and a portion of a heavy chain, and a protein linked to the portion of the heavy chain. In some embodiments, a reporter is conjugated to at least one of the lysine or cysteine residues. In other embodiments, a drug molecule is conjugated to at least one of the lysine or cysteine residues. The lysine or cysteine residues throughout the protein are space such that when a reporter (or drug molecule) is conjugated to a lysine or cysteine residue, one reporter (or drug molecule) does not interfere with the conjugation of another reporter (or drug molecule).

The present invention may also feature an isolated antibody composition comprising at least one variable region (Fab) comprising a light chain and a portion of a heavy chain (e.g., a full length heavy chain or a portion thereof), and a protein linked to the portion of the heavy chain, wherein the protein comprises one or more repeats of an epitope for a single-domain antibody (sdAb) fragment or a single-chain variable fragment (scFv) to bind. In some embodiments, the sdAb or the scFV is conjugated to a reporter or a drug molecule.

In some embodiments, the reporter is a fluorescent reporter (e.g., a fluorescent dye). In other embodiments, the reporter is an enzymatic reporter (e.g., horseradish peroxidase or alkaline phosphatase).

The isolated antibody composition described herein allows for multiplexing immunoassays. In some embodiments, isolated antibody compositions are for direct immunoassays. In other embodiments, the isolated antibody composition is for indirect immunoassays. Non-limiting examples of immunoassays include but are not limited to enzyme-linked immunosorbent assay (ELISA), immunohistochemistry (IHC), Immunocytochemistry (ICC), Immunofluorescence assay (IFA), or Flow Cytometry (FACS).

The present invention features an isolated antibody composition. The isolated antibody composition may comprise a heavy chain comprising one or more amino acid modifications (substitutions, deletions, or addition) In some embodiments, at least one of the one or more amino acid modifications comprises an amino acid substituted to a lysine or cysteine residue; wherein a conjugation occurs at at least one of lysine or cysteine residues, and wherein naturally occurring and substituted lysine or cysteine residues throughout the heavy chain are spaced such that when a there is a conjugation to a lysine or cysteine residue it has limited interference with conjugations on another lysine or cysteine; and a light chain comprising one or more amino acid modifications (substitutions, deletions, or addition), where, in some embodiment, at least one of the one or more amino acids substitutions comprises an amino acid substituted to a lysine or cysteine residue.

In some embodiments, the isolated antibody composition may comprise a heavy chain; and a light chain comprising one or more amino acid modifications (substitutions, deletions, or addition). In some embodiments, at least one of the one or more amino acid modifications comprises an amino acid substituted to a lysine or cysteine residue.

In other embodiments, the isolated antibody composition may comprise a heavy chain comprising one or more amino acid modifications (substitutions, deletions, or additions). In some embodiments, at least one of the one or more amino acid modifications comprises an amino acid substituted to a lysine or cysteine residue, wherein a conjugation occurs at at least one of lysine or cysteine residues, and wherein naturally occurring and substituted lysine or cysteine residues throughout the heavy chain are spaced such that when a there is a conjugation to a lysine or cysteine residue it has limited interference with conjugations on another lysine or cysteine; and a light chain.

Is some embodiments, the one of the one or more amino acid substitutions in the heavy chain or the light chain comprises a naturally occurring lysine or cysteine residue substituted to a conserved amino acid residue; wherein the conserved amino acid residue allows for complementary function but no conjugation.

In some embodiments, the one or more modifications result in a single amino acid substitution. In other embodiments, the one or more modifications result in a single amino acid insertion. In further embodiments, the one or more modifications result in a single amino acid deletion.

Various embodiments described herein may be combined to generate isolated antibody compositions in accordance with the present invention.

### Embodiments Set A

The following embodiments are intended to be illustrative only and not to be limiting in any way.

Embodiment 1A: An engineered binder composition comprising: a) a primary target binder, wherein the primary target binder is configured to directly bind a target molecule; and b) one or a combination of: (i) a reporter conjugated to at least one conjugation-targeted amino acid residue in the primary target binder; (ii) a drug conjugated to at least one conjugation-targeted amino acid residue in the primary target binder; a reporter conjugated to at least one post-translational modification (e.g., a glycosylation moiety) on the primary target binder; or a drug conjugated to at least one post-translational modification (e.g., a glycosylation moiety) on the primary target binder.

Embodiment 2A: The composition of embodiment 1A, wherein the conjugation-targeted amino acid residues throughout the primary binder are spaced such that when the reporter is conjugated to the conjugation-targeted amino acid residue one reporter does not interfere with the conjugation of another reporter.

Embodiment 3A: The composition embodiment 1A or embodiment 2A, wherein the conjugation-targeted amino acid residue comprises cystine, lysine or a combination thereof.

Embodiment 4A: The composition of any one of embodiments 1A-3A, wherein the primary target binder comprises an antibody or a fragment thereof.

Embodiment 5A: The composition of embodiment 4A, wherein the antibody fragment comprises a fragment antigen-binding region (Fab region), F(ab′)2, Fab′, Fv (Fragment Variable), single-domain antibody (sdAb) fragment or a single-chain variable fragment (scFv).

Embodiment 6A: The composition of any one of embodiments 1A-3A, wherein the primary target binder comprising synthetic binders or engineered binders

Embodiment 7A: The composition of embodiment 6A, wherein the synthetic binder comprises an aptamer, affibody, Designed Ankyrin Repeat Protein (DARPins), or molecularly imprinted polymer.

Embodiment 8A: The composition of any one of embodiments 1A-7A, wherein the reporter is a fluorescence reporter, fluorochrome, or an enzyme reporter.

Embodiment 9A: The composition of embodiment 8A, wherein the enzyme reporter comprises a chromogen such as 3,3′-diaminobenzidine tetrahydrochloride (DAB), horseradish peroxidase (HRP) or alkaline phosphatase (AP).

Embodiment 10A: The composition of embodiment 8A, wherein the fluorescence reporter is a fluorescence dye.

Embodiment 11A: The composition of any one of embodiments 1A-7A, wherein the reporter is an oligonucleotide.

Embodiment 12A: The composition of anyone of claims 1A-7A, wherein the reporter is a metal isotope.

Embodiment 13A: The composition of any one of embodiments 1A-12A, further comprising a primary fusion component recombinantly linked to the primary target binder.

Embodiment 14A: The composition of embodiment 13A, wherein the composition comprises two or more primary fusion components, wherein the two or more primary fusion components are recombinantly linked to the primary target binder in tandem.

Embodiment 15A: The composition of embodiment 13A or embodiment 14A, wherein the primary fusion is recombinately linked to an N or C termini of the primary target binder.

Embodiment 16A: The composition of embodiment 13A or embodiment 14A, wherein the primary fusion component is recombinantly linked to an internal portion of the primary target binder.

Embodiment 17A: The composition of any one of embodiments 13A-16A, wherein the primary fusion component is a binding component that allows binding of a secondary binder to the primary target binder.

Embodiment 18A: The composition of embodiment 17A, wherein the primary fusion component comprises a protein, an epitope, a pseudo-epitope, or a combination thereof.

Embodiment 19A: The composition of any one of embodiments 13A-16A, wherein the primary fusion component is a secondary binder.

Embodiment 20A: The composition of any one of embodiments 1A-12A, further comprising a secondary binder (e.g., when a primary fusion component is a secondary binder) bound directly to the primary target binder.

Embodiment 21A: The composition of any one of embodiments 17A-20A, wherein the secondary binder comprises antibody or a fragment thereof, wherein the antibody fragment comprises a fragment antigen-binding region (Fab region), F(ab′)2, Fab′, Fv (Fragment Variable), single-domain antibody (sdAb) fragment or a single-chain variable fragment (scFv).

Embodiment 22A: The composition of any one of embodiments 17A-20A, wherein the secondary binder comprises synthetic binders or engineered binders; wherein the synthetic binder comprises an aptamer, affibody, Designed Ankyrin Repeat Protein (DARPins), or molecularly imprinted polymer.

Embodiment 23A: The composition of any one of embodiments 17A-22A, wherein the secondary binder further comprises a reporter conjugated to at least one conjugation-targeted amino acid residue in the secondary binder.

Embodiment 24A: The composition of embodiment 23A, wherein the conjugation-targeted amino acid residues throughout the secondary binder are spaced such that when the reporter is conjugated to the conjugation-targeted amino acid residue one reporter does not interfere with the conjugation of another reporter.

Embodiment 25A: The composition of embodiment 23A or embodiment 24A, wherein the conjugation-targeted amino acid residue comprises cystine, lysine or a combination thereof.

Embodiment 26A: The composition of any one of embodiments 17A-25A, wherein the reporter is a fluorescence reporter, fluorochrome, an enzyme reporter, an oligonucleotide, or a metal isotope.

Embodiment 27A: The composition of any one of embodiments 17A-26A, wherein the secondary binder further comprises a secondary fusion component attached to the primary target binder.

Embodiment 28A: The composition of embodiment 27A, wherein the secondary binder further comprises two or more secondary fusion components, wherein the two or more secondary fusion components are recombinantly linked to the secondary binder in tandem.

Embodiment 29A: The composition of embodiment 27A or embodiment 28A, wherein the secondary fusion component is recombinately linked to an N or C termini of the secondary binder.

Embodiment 30A: The composition of embodiment 27A or embodiment 28A, wherein the secondary fusion component is recombinantly linked to an internal portion of the secondary binder.

Embodiment 31A: The composition of any one of embodiments 27A-30A, wherein the secondary fusion component is a binding component that allows binding of a tertiary binder to the secondary binder.

Embodiment 32A: The composition of embodiment 31A, wherein the binding component comprises a protein, an epitope, a pseudo-epitope, or a combination thereof.

Embodiment 33A: The composition of any one of embodiments 27A-32A, wherein the secondary fusion component is a tertiary binder.

Embodiment 34A: The composition of embodiment 27A or embodiment 28A, further comprising a tertiary binder (e.g., when a secondary fusion component is a tertiary binder) bound directly to the secondary binder.

Embodiment 35A: The composition of any one of embodiments 31A-34A, wherein the tertiary binder comprises antibody or a fragment thereof, wherein the antibody fragment comprises a fragment antigen-binding region (Fab region), F(ab′)2, Fab′, Fv (Fragment Variable), single-domain antibody (sdAb) fragment or a single-chain variable fragment (scFv).

Embodiment 36A: The composition of any one of embodiments 31A-34A, wherein the tertiary binder comprises synthetic binders or engineered binders; wherein the synthetic binder comprises an aptamer, affibody, Designed Ankyrin Repeat Protein (DARPins), or molecularly imprinted polymer.

Embodiment 37A: The composition of any one of embodiments 31A-36A, wherein the tertiary binder further comprises a reporter conjugated to at least one conjugation-targeted amino acid residue in the tertiary binder.

Embodiment 38A: The composition of embodiment 37A, wherein the conjugation-targeted amino acid residues throughout the tertiary binder are spaced such that when the reporter is conjugated to the conjugation-targeted amino acid residue one reporter does not interfere with the conjugation of another reporter.

Embodiment 39A: The composition of embodiment 37A or embodiment 38A, wherein the conjugation-targeted amino acid residue comprises cystine, lysine or a combination thereof.

Embodiment 40A: The composition of any one of embodiments 37A-39A, wherein the reporter is a fluorescence reporter, fluorochrome, an enzyme reporter, an oligonucleotide, or a metal isotope.

Embodiment 41A: The composition of any one of embodiments 37A-26A, wherein the tertiary binder further comprises a tertiary fusion component attached (e.g., recombinantly linked or bound) to the tertiary binder.

Embodiment 42A: The composition of embodiment 41A, wherein the tertiary binder further comprises two or more tertiary fusion components, wherein the two or more tertiary fusion components are recombinantly linked to the tertiary binder in tandem.

Embodiment 43A: The composition of embodiment 41A or embodiment 42A, wherein the tertiary fusion component is recombinately linked to an N or C termini of the tertiary binder.

Embodiment 44A: The composition of embodiment 41A or embodiment 42A, wherein the tertiary fusion component is recombinantly linked to an internal portion of the tertiary binder.

Embodiment 45A: The composition of any one of embodiments 41A-44A, wherein the tertiary fusion component is a binding component that allows binding of a quaternary binder to the tertiary binder.

Embodiment 46A: The composition of embodiment 45A, wherein the binding component comprises a protein, an epitope, a pseudo-epitope, or a combination thereof.

Embodiment 47A: The composition of any one of embodiments 41A-46A, wherein the secondary fusion component is a tertiary binder.

### Embodiments Set B

The following embodiments are intended to be illustrative only and not to be limiting in any way.

Embodiment 1B: An isolated antibody composition comprising: a) a constant region (Fc), wherein the constant region comprises one or more amino acids substitutions, wherein at least one of the one or more amino acids substitutions comprises an amino acid substituted to a lysine or cysteine residue; wherein a conjugation occurs at at least one of lysine or cysteine residues, and wherein naturally occurring and substituted lysine or cysteine residues throughout the constant region are spaced such that when a there is a conjugation to a lysine or cysteine residue it has limited interference with conjugations on another lysine or cysteine; and b) a variable region (Fab), wherein the variable region comprises one or more amino acids substitutions, wherein at least one of the one or more amino acids substitutions comprises an amino acid substituted to a lysine or cysteine residue.

Embodiment 2B: An isolated antibody composition comprising: a) a constant region (Fc); and b) a variable region (Fab), wherein the variable region comprises one or more amino acids substitutions, wherein at least one of the one or more amino acids substitutions comprises an amino acid substituted to a lysine or cysteine residue.

Embodiment 3B: An isolated antibody composition comprising: a) a constant region (Fc), wherein the constant region comprises one or more amino acids substitutions, wherein at least one of the one or more amino acids substitutions comprises an amino acid substituted to a lysine or cysteine residue; wherein a conjugation occurs at at least one of lysine or cysteine residues, and wherein naturally occurring and substituted lysine or cysteine residues throughout the constant region are spaced such that when a there is a conjugation to a lysine or cysteine residue it has limited interference with conjugations on another lysine or cysteine; and b) a variable region (Fab).

Embodiment 4B: The composition of any one of embodiments 1B-3B, wherein the one of the one or more amino acid substitutions in the constant region or the variable region comprises a naturally occurring lysine or cysteine residue substituted to a conserved amino acid residue; wherein the conserved amino acid residue allows for complementary function but no conjugation.

Embodiment 5B: The composition of any one of embodiments 1B-4B further comprising: a) an additional polypeptide sequence with exposed lysines or cysteines for conjugation added to a C-terminus of a heavy chain; b) an additional polypeptide sequence with exposed lysines or cysteines for conjugation added to a C-terminus of a light chain, or a combination thereof.

Embodiment 6B: The composition of any one of embodiments 1B-4B further comprising: a) an additional polypeptide sequence of an AB-fragment such as a single-domain antibody (sdAb) fragment or a single-chain variable fragment (scFv) added to a C-terminus of a heavy chain; b) an additional polypeptide sequence of an scFv or sdAB added to a C-terminus of a light chain; or a combination thereof; wherein the additional polypeptide sequence is able to bind to a conjugated protein with the corresponding epitope(s) for the said additional polypeptide sequence of the scFv or sdAB.

Embodiment 7B: The composition of any one of embodiments 1B-4B further comprising: a) an additional polypeptide sequence with one or more epitopes to a C-terminus of a heavy chain; b) an additional polypeptide sequence with one or more epitopes added to a C-terminus of the light chain; or a combination thereof, wherein the additional polypeptide sequence epitopes are able to be bound to by a corresponding single-domain antibody (sdAb) fragment, a single-chain variable fragment (scFv), or an antibody (Ab).

Embodiment 8B: A composition comprising a single-domain antibody (sdAb) fragment or a single-chain variable fragment (scFv); and at least one of: a) a polypeptide sequence; b) a polypeptide sequence of one or more additional scFv or sdAB; wherein the scFV or sdAB is conjugated itself and/or bound to a conjugated protein with the corresponding epitope(s) for the added scFv or sdAb.

Embodiment 9B: The composition of any one of embodiments 1B-8B, wherein a reporter or a drug is conjugated to at least one of the lysine or cysteine residues.

Embodiment 10B: An isolated antibody composition comprising: a) a constant region (Fc), wherein the constant region comprises one or more amino acids substitutions, wherein at least one of the one or more amino acids substitutions comprises an amino acid substituted to a lysine residue; wherein a reporter is conjugated to at least one of lysine residues, and wherein naturally occurring and substituted lysine residues throughout the constant region are spaced such that when the reporter is conjugated to a lysine residue one reporter does not interfere with the conjugation of another reporter; and b) a variable region (Fab).

Embodiment 11B: The composition of embodiment 10B, wherein the variable region (Fab) comprises one or more amino acids substitutions, wherein at least one of the one or more amino acids substitutions comprises an amino acid substituted to a lysine residue.

Embodiment 12B: The composition of embodiment 10B or embodiment 11B, wherein one of the one or more amino acid substitutions in the constant region or the variable region (Fab) comprises a naturally occurring lysine residue substituted to a conserved amino acid residue, wherein the conserved amino acid residue allows for complementary function but no conjugation of a reporter.

Embodiment 13B: An isolated antibody composition comprising: a) a constant region (Fc), wherein the constant region comprises one or more amino acids substitutions, wherein at least one of the one or more amino acids substitutions comprises an amino acid substituted to a cysteine residue; wherein a reporter conjugated to at least one of the cysteine residues, and wherein naturally occurring and modified cysteines throughout the constant region are space such that when the reporter is conjugated to a cysteine residue one reporter does not interfere with the conjugation of another reporter; and b) a variable region (Fab).

Embodiment 14B: The composition of embodiment 13B, wherein the variable region comprises one or more amino acids substitutions, wherein at least one of the one or more amino acids substitutions comprises an amino acid substituted to a cysteine residue.

Embodiment 15B: The composition of embodiment 13B or embodiment 14B, wherein the one of the one or more amino acid substitutions in the constant region or the variable region comprises a naturally occurring cysteine residue substituted to a conserved amino acid residue; wherein the conserved amino acid residue allows for complementary function but no conjugation of a reporter.

Embodiment 16B: The composition of any one of embodiments 10B-15B further comprising a polypeptide sequence linked to a heavy chain or a light chain or the constant region or variable region.

Embodiment 17B: The composition of embodiment 16B, wherein a reporter is conjugate to at least one residue of the protein, wherein the reporter is conjugated to at least one cysteine residue of the protein; wherein the reporter is conjugated to at least one cysteine residue to the protein.

Embodiment 18B: An isolated antibody composition comprising: a) at least one variable region (Fab) comprising a light chain and a portion of a heavy chain, and b) a protein is recombinantly linked to the portion of the heavy chain; wherein the protein comprises one or more amino acid substitutions, wherein at least one of the one or more amino acid substitutions comprises an amino acid substituted to a lysine residue; wherein a reporter is conjugated to at least one of the lysine residues, and wherein naturally occurring and substituted lysine residues throughout the protein are space such that when a reporter is conjugated to a lysine residue one reporter does not interfere with the conjugation of another reporter.

Embodiment 19B: An isolated antibody composition comprising: a) at least one single-domain antibody (sdAb) fragment or one single-chain variable fragment (scFv), and b) protein is recombinantly linked to the portion of the heavy chain; wherein the protein comprises one or more amino acid substitutions, wherein at least one of the one or more amino acid substitutions comprises an amino acid substituted to a lysine residue; wherein a reporter is conjugated to at least one of the lysine residues, and wherein naturally occurring and substituted lysine residues throughout the protein are space such that when a reporter is conjugated to a lysine residue one reporter does not interfere with the conjugation of another reporter.

Embodiment 20B: The composition of embodiment 18B or embodiment 19B, wherein one of the one or more amino acid substitutions in the protein comprises a naturally occurring lysine residue substituted to a conserved amino acid residue; wherein the conserved amino acid residue allows for complementary function but no conjugation of a reporter.

Embodiment 21B: An isolated antibody composition comprising: a) at least one variable region (Fab) comprising a light chain and a portion of a heavy chain; and b) a protein is recombinantly linked to the portion of the heavy chain; wherein the protein comprises one or more amino acid substitutions, wherein at least one of the one or more amino acid substitutions comprises an amino acid substituted to a cysteine residue; wherein a reporter is conjugated to at least one of the cysteine residues, and wherein naturally occurring and substituted cysteine residues throughout the protein are space such that when a reporter is conjugated to a cysteine residue one reporter does not interfere with the conjugation of another reporter.

Embodiment 22B: An isolated antibody composition comprising: a) at least one single-domain antibody (sdAb) fragment or one single-chain variable fragment (scFv), and b) a protein is recombinantly linked to the portion of the heavy chain; wherein the protein comprises one or more amino acid substitutions, wherein at least one of the one or more amino acid substitutions comprises an amino acid substituted to a cysteine residue; wherein a reporter is conjugated to at least one of the cysteine residues, and wherein naturally occurring and substituted cysteine residues throughout the protein are space such that when a reporter is conjugated to a cysteine residue one reporter does not interfere with the conjugation of another reporter.

Embodiment 23B: The composition of embodiment 21B or embodiment 22B, the one of the one or more amino acid substitutions in the constant region comprises a naturally occurring cysteine residue substituted to a conserved amino acid residue; wherein the conserved amino acid residue allows for complementary function but no conjugation of a reporter.

Embodiment 24B: An isolated antibody composition comprising: a) at least one variable region (Fab) comprising a light chain and a portion of a heavy chain; and b) a protein is recombinantly linked to the portion of the heavy chain; wherein a reporter is conjugated to at least one of the lysine residues or cysteine residues, and the lysine residues throughout the protein are space such that when a reporter is conjugated to a lysine residue one reporter does not interfere with the conjugation of another reporter.

Embodiment 25B: An isolated antibody composition comprising: a) at least one single-domain antibody (sdAb) fragment or one single-chain variable fragment (scFv); and b) a protein is recombinantly linked to the sdAb or scFv; wherein a reporter is conjugated to at least one of the lysine residues, and the lysine residues throughout the protein are space such that when a reporter is conjugated to a lysine residue one reporter does not interfere with the conjugation of another reporter.

Embodiment 26B: An isolated antibody composition comprising: a) at least one variable region (Fab) comprising a light chain and a portion of a heavy chain; and b) a protein is recombinantly linked to the portion of the heavy chain; wherein a reporter is conjugated to at least one of the cysteine residues, and the cysteine residues throughout the protein are space such that when a reporter is conjugated to a cysteine residue one reporter does not interfere with the conjugation of another reporter.

Embodiment 27B: An isolated antibody composition comprising: a) at least one single-domain antibody (sdAb) fragment or one single-chain variable fragment (scFv); and b) a protein is recombinantly linked to the portion of the sdAb or scFv; wherein a reporter is conjugated to at least one of the cysteine residues, and the cysteine residues throughout the protein are space such that when a reporter is conjugated to a cysteine residue one reporter does not interfere with the conjugation of another reporter

Embodiment 28B: An isolated antibody composition comprising: a) at least one variable region (Fab) comprising a light chain and a portion of a heavy chain, at least one single-domain antibody (sdAb) fragment or at least one single-chain variable fragment (scFv); and b) a protein is recombinantly linked to the portion of the heavy chain; wherein the protein comprises one or more repeats of an epitope for a single-domain antibody (sdAb) fragment or a single-chain variable fragment (scFv) to bind, wherein the sdAb or the scFV is conjugated to a reporter.

Embodiment 29B: The composition of any one of embodiments 1B-28B, wherein the reporter is a fluorescence reporter or an enzyme reporter.

Embodiment 30B: The composition of embodiment 29B, wherein the enzyme reporter is horseradish peroxidase (HRP) or alkaline phosphatase (AP).

Embodiment 31B: The composition of embodiment 29B, wherein the fluorescence reporter is a fluorescence dye.

Embodiment 32B: The composition of any one of embodiments 1B-29B, wherein the isolated antibody composition allows for multiplexing immunoassays.

Embodiment 33B: The composition of embodiment 32B, wherein the isolated antibody composition is for direct immunoassays.

Embodiment 34B: The composition of embodiment 32B, wherein the isolated antibody composition is for indirect immunoassays.

Embodiment 35B: The composition of embodiment 33B or embodiment 34B, wherein the immunoassays comprise enzyme-linked immunosorbent assay (ELISA), immunohistochemistry (IHC), Immunocytochemistry (ICC), Immunofluorescence assay (IFA), or Flow Cytometry (FACS).

Embodiment 36B: An isolated antibody composition comprising: a) a constant region (Fc), wherein the constant region comprises one or more amino acids substitutions, wherein at least one of the one or more amino acids substitutions comprises an amino acid substituted to a lysine or cysteine residue; wherein a drug molecule is conjugated to at least one of lysine or cysteine residues, and wherein naturally occurring and substituted lysine or cysteine residues throughout the constant region are space such that when the drug molecule is conjugated to a lysine or cysteine residue one drug molecule does not interfere with the conjugation of another drug molecule; and b) a variable region (Fab), wherein the variable region comprises one or more amino acids substitutions, wherein at least one of the one or more amino acids substitutions comprises an amino acid substituted to a lysine or cysteine residue.

Embodiment 37B: An isolated antibody composition comprising: a) a constant region (Fc), wherein the constant region comprises one or more amino acids substitutions, wherein at least one of the one or more amino acids substitutions comprises an amino acid substituted to a lysine or cysteine residue; wherein a drug molecule is conjugated to at least one of lysine or cysteine residues, and wherein naturally occurring and substituted lysine or cysteine residues throughout the constant region are space such that when the drug molecule is conjugated to a lysine or cysteine residue one drug molecule does not interfere with the conjugation of another drug molecule; and b) a variable region (Fab).

Embodiment 38B: The composition of embodiment 37B, wherein the variable region (Fab) comprises one or more amino acids substitutions, wherein at least one of the one or more amino acids substitutions comprises an amino acid substituted to a lysine or cysteine residue.

Embodiment 39B: The composition of any one of embodiments 36B-38B, wherein the one of the one or more amino acid substitutions in the constant region or variable region comprises a naturally occurring lysine or cysteine residue substituted to a conserved amino acid residue; wherein the conserved amino acid residue allows for complementary function but no conjugation of a drug molecule.

Embodiment 40B: An isolated antibody composition comprising: a) at least one variable region (Fab) comprising a light chain and a portion of a heavy chain; and b) a protein conjugated to the portion of the heavy chain; wherein the protein comprises one or more amino acid substitutions, wherein at least one of the one or more amino acid substitutions comprises an amino acid substituted to a lysine or cysteine residue; wherein a drug molecule is conjugated to at least one of the lysine or cysteine residues, and wherein naturally occurring and substituted lysine or cysteine residues throughout the protein are space such that when a drug molecule is conjugated to a lysine or cysteine residue one drug molecule does not interfere with the conjugation of another drug molecule.

Embodiment 41B: An isolated antibody composition comprising: a) at least one single-domain antibody (sdAb) fragment, or at least a single-chain variable fragment (scFv); and b) a protein conjugated to the portion of the sdAb or scFv; wherein the protein comprises one or more amino acid substitutions, wherein at least one of the one or more amino acid substitutions comprises an amino acid substituted to a lysine or cysteine residue; wherein a drug molecule is conjugated to at least one of the lysine or cysteine residues, and wherein naturally occurring and substituted lysine or cysteine residues throughout the protein are space such that when a drug molecule is conjugated to a lysine or cysteine residue one drug molecule does not interfere with the conjugation of another drug molecule.

Embodiment 42B: The composition of embodiment 40B or embodiment 41B, the one of the one or more amino acid substitutions in the constant region comprises a naturally occurring cysteine residue substituted to a conserved amino acid residue; wherein the conserved amino acid residue allows for complementary function but no conjugation of a drug molecule.

Embodiment 43B: An isolated antibody composition comprising: a) at least one variable region (Fab) comprising a light chain and a portion of a heavy chain; and b) a protein conjugated to the portion of the heavy chain; wherein a drug molecule is conjugated to at least one of the lysine or cysteine residues, and the lysine or cysteine residues throughout the protein are space such that when a drug is conjugated to a lysine or cysteine residue one drug molecule does not interfere with the conjugation of another drug molecule.

Embodiment 44B: An isolated antibody composition comprising: a) at least one single-domain antibody (sdAb) fragment, or at least a single-chain variable fragment (scFv); and b) a protein conjugated to the portion of the sdAb or scFv; wherein a drug molecule is conjugated to at least one of the lysine or cysteine residues, and the lysine or cysteine residues throughout the protein are space such that when a drug is conjugated to a lysine or cysteine residue one drug molecule does not interfere with the conjugation of another drug molecule.

Embodiment 45B: An isolated antibody composition comprising: a) a heavy chain comprising one or more amino acid substitutions, wherein at least one of the one or more amino acid substitutions comprises an amino acid substituted to a lysine or cysteine residue; wherein a conjugation occurs at at least one of lysine or cysteine residues, and wherein naturally occurring and substituted lysine or cysteine residues throughout the heavy chain are spaced such that when a there is a conjugation to a lysine or cysteine residue it has limited interference with conjugations on another lysine or cysteine; and b) a light chain comprising one or more amino acids substitutions, wherein at least one of the one or more amino acids substitutions comprises an amino acid substituted to a lysine or cysteine residue.

Embodiment 46B: An isolated antibody composition comprising: a) a heavy chain; and b) a light chain comprising one or more amino acid substitutions, wherein at least one of the one or more amino acid substitutions comprises an amino acid substituted to a lysine or cysteine residue.

Embodiment 47B: An isolated antibody composition comprising: a) a heavy chain comprising one or more amino acid substitutions, wherein at least one of the one or more amino acid substitutions comprises an amino acid substituted to a lysine or cysteine residue; wherein a conjugation occurs at at least one of lysine or cysteine residues, and wherein naturally occurring and substituted lysine or cysteine residues throughout the heavy chain are spaced such that when a there is a conjugation to a lysine or cysteine residue it has limited interference with conjugations on another lysine or cysteine; and b) a light chain.

Embodiment 48B: The composition of any one of embodiments 45B-48B, wherein the one of the one or more amino acid substitutions in the heavy chain or the light chain comprises a naturally occurring lysine or cysteine residue substituted to a conserved amino acid residue; wherein the conserved amino acid residue allows for complementary function but no conjugation.

Embodiment 49B: The composition of any one of embodiments 45B-48B further comprising: a) an additional polypeptide sequence with exposed lysines or cysteines for conjugation added to a C-terminus of the heavy chain; b) an additional polypeptide sequence with exposed lysines or cysteines for conjugation added to a C-terminus of the light chain, or a combination thereof.

Embodiment 50B: The composition of any one of embodiments 45B-48B further comprising: a) an additional polypeptide sequence of a single-domain antibody (sdAb) fragment or a single-chain variable fragment (scFv) added to a C-terminus of the heavy chain; b) an additional polypeptide sequence of an scFv or sdAB added to a C-terminus of the light chain; or a combination thereof, wherein the additional polypeptide sequence can bind to a conjugated protein with the corresponding epitope(s) for the said additional polypeptide sequence of the scFv or sdAB.

Embodiment 51B: The composition of any one of embodiments 45B-48B further comprising: a) an additional polypeptide sequence with one or more epitopes to a C-terminus of the heavy chain; b) an additional polypeptide sequence with one or more epitopes added to a C-terminus of the light chain; or a combination thereof, wherein the additional polypeptide sequence epitopes can be bound to by a corresponding single-domain antibody (sdAb) fragment, a single-chain variable fragment (scFv), or an antibody (Ab).

Embodiment 52B: The composition of any one of embodiments 45B-51B, wherein a reporter or a drug is conjugated to at least one of the lysine or cysteine residues.

Embodiment 53B: An isolated antibody composition comprising: a) an antibody (AB) fragment; and b) a protein is recombinantly linked to the AB fragment; wherein a reporter (or drug molecule) is conjugated to at least one of the lysine residues, and the lysine residues throughout the protein are spaced such that when a reporter (or drug molecule) is conjugated to a lysine residue one reporter (or drug molecule) does not interfere with the conjugation of another reporter (or drug molecule).

Embodiment 54B: An isolated antibody composition comprising: a) an antibody (AB) fragment; and b) a protein is recombinantly linked to the portion of the AB fragment; wherein a reporter (or drug molecule) is conjugated to at least one of the cysteine residues, and the cysteine residues throughout the protein are spaced such that when a reporter (or drug molecule) is conjugated to a cysteine residue one reporter (or drug molecule) does not interfere with the conjugation of another reporter (or drug molecule).

Embodiment 55B: The composition of embodiment 53B or embodiment 54B, wherein the AB fragment comprises a fragment antigen-binding region (Fab region), F(ab′)2, Fab′, Fv (Fragment Variable), single-domain antibody (sdAb) fragment or a single-chain variable fragment (scFv).

Embodiment 56B: An isolated antibody composition comprising: a) a synthetic binder; and b) a protein is recombinantly linked to the synthetic binder wherein a reporter (or drug molecule) is conjugated to at least one of the lysine residues, and the lysine residues throughout the protein are spaced such that when a reporter (or drug molecule) is conjugated to a lysine residue one reporter (or drug molecule) does not interfere with the conjugation of another reporter (or drug molecule).

Embodiment 57B: An isolated antibody composition comprising: a) a synthetic binder; and b) a protein is recombinantly linked to the portion of the synthetic binder; wherein a reporter (or drug molecule) is conjugated to at least one of the cysteine residues, and the cysteine residues throughout the protein are spaced such that when a reporter (or drug molecule) is conjugated to a cysteine residue one reporter (or drug molecule) does not interfere with the conjugation of another reporter (or drug molecule).

Embodiment 58B: The composition of embodiment 56B or embodiment 57B, wherein the synthetic binder comprises an aptamer, affibody, Designed Ankyrin Repeat Protein (DARPins), or molecularly imprinted polymer.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An synthetic binder composition comprising:
a) a primary target binder, wherein the primary target binder is configured to directly bind a target;
b) a primary fusion component to attached to the primary target binder; wherein the primary fusion component is either (i) a binding component that allows binding of a secondary binder to the primary target binder or (ii) a secondary binder; and
c) one or a combination of:
i) a reporter conjugated to at least one conjugation-targeted amino acid residue in either one or both of (i) the primary target binder or (ii) the primary fusion component;
ii) a drug conjugated to at least one conjugation-targeted amino acid residue in either one or both of (i) the primary target binder or (ii) the primary fusion component;
iii) a reporter conjugated to at least one post-translational modification on one or both of (i) the primary target binder or (ii) the primary fusion component; or
iv) a drug conjugated to at least one post-translational modification on one or both of (i) the primary target binder or (ii) the primary fusion component.

2. The composition of claim 1, wherein the primary target binder comprises an antibody or a fragment thereof.

3. The composition of claim 2, wherein the antibody fragment comprises a fragment antigen-binding region (Fab region), F(ab′)2, Fab′, Fv (Fragment Variable), single-domain antibody (sdAb) fragment or a single-chain variable fragment (scFv).

4. The composition of claim 1, wherein the primary target binder comprises synthetic binders or engineered binders.

5. The composition of claim 4, wherein the synthetic binder comprises an aptamer, affibody, Designed Ankyrin Repeat Protein (DARPins), or molecularly imprinted polymer.

6. The composition of claim 1, wherein the primary fusion component is recombinantly linked to the primary target binder.

7. The composition of claim 6, wherein the primary fusion component is recombinantly linked to an N or C termini of the primary target binder.

8. The composition of claim 6, wherein the primary fusion component is recombinantly linked to an internal portion of the primary target binder.

9. The composition of claim 6, wherein the composition comprises two or more primary fusion components, wherein the two or more primary fusion components are recombinantly linked to the primary target binder in tandem.

10. The composition of claim 1, wherein when the primary fusion component is a secondary binder, the primary fusion component binds directly to the primary target binder.

11. The composition of claim 1, wherein the binding component comprises a protein, an epitope, a pseudo-epitope or a combination thereof.

12. The composition of claim 1, wherein the conjugation-targeted amino acid residues throughout the primary binder and/or the primary fusion component are spaced such that when the reporter is conjugated to the conjugation-targeted amino acid residue one reporter does not interfere with the conjugation of another reporter.

13. The composition of claim 1, wherein the conjugation-targeted amino acid residue comprises cystine, lysine or a combination thereof.

14. The composition of claim 1, wherein the reporter is a fluorescence reporter, fluorochrome, or an enzyme reporter.

15. The composition of claim 14, wherein the enzyme reporter comprises a chromogen such as 3,3′-diaminobenzidine tetrahydrochloride (DAB), horseradish peroxidase (HRP) or alkaline phosphatase (AP).

16. The composition of claim 14, wherein the fluorescence reporter is a fluorescence dye.

17. The composition of claim 1, wherein the reporter is an oligonucleotide.

18. The composition of claim 1, wherein the reporter is a metal isotope.

19. The composition of claim 1, wherein the secondary binder comprises antibody or a fragment thereof, wherein the antibody fragment comprises a fragment antigen-binding region (Fab region), F(ab′)2, Fab′, Fv (Fragment Variable), single-domain antibody (sdAb) fragment or a single-chain variable fragment (scFv).

20. The composition of claim 1, wherein the secondary binder comprises synthetic binders or engineered binders; wherein the synthetic binder comprises an aptamer, affibody, Designed Ankyrin Repeat Protein (DARPins), or molecularly imprinted polymer.
